SEQUENCE LISTING (13 sequences)

SEQ ID NO: 1 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 2 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 3 - protein, 29 aa, ARTIFICIAL SEQUENCE
  Biotinylated CCR4 peptide
MNPTDIADTTLDESIYSNYYLYESIPKPK

SEQ ID NO: 4 - protein, 31 aa, ARTIFICIAL SEQUENCE
  Biotinylated CCR4 peptide
MNPTDIADTTLDESIYSNYYLYESIPKPCTK

SEQ ID NO: 5 - protein, 28 aa, ARTIFICIAL SEQUENCE
  Biotinylated CD20 peptide
KGGYNCEPANPSEKNSPSTQYCYSIQSL

SEQ ID NO: 6 - protein, 27 aa, ARTIFICIAL SEQUENCE
  Biotinylated CD20 peptide
YNCEPANPSEKNSPSTQYCYSIQSLKK

SEQ ID NO: 7 - protein, 29 aa, ARTIFICIAL SEQUENCE
  Biotinylated CD20 peptide (MBT4736)
XKGGYNCEPANPSEKNSPSTQYCYSIQSL

SEQ ID NO: 8 - protein, 28 aa, ARTIFICIAL SEQUENCE
  Biotinylated CD20 peptide (MBT4737)
YNCEPANPSEKNSPSTQYCYSIQSLKXK

SEQ ID NO: 9 - DNA, 66 bp, ARTIFICIAL SEQUENCE
  VK-1 signal peptide coding sequence
atggacatgagggtgcccgctcagctcctggggctcctgctgctgtggctgagaggtgcgcgctgt

SEQ ID NO: 10 - protein, 22 aa, ARTIFICIAL SEQUENCE
  Synthetic Construct
MDMRVPAQLLGLLLLWLRGARC

SEQ ID NO: 11 - protein, 254 aa, Homo sapiens
  MISC_FEATURE: CD16a
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 12 - protein, 192 aa, Homo sapiens
  MISC_FEATURE: G17-Q208 fragment of human CD16a
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 13 - protein, 1255 aa, Homo sapiens [sequence omitted]

(19) United States
(12) Patent Application Publication
(10) Pub. No.: US 2014/0072983 A1
(43) Pub. Date: Mar. 13, 2014

(54) Dual function in vitro target binding assay for the detection of neutralizing antibodies against target antibodies

(71) Applicants: Xinyi Cynthia Chen, Calabasas, CA (US); Shalini Gupta, Newbury Park, CA (US); Francesca Civoli, Newbury Park, CA (US)

(72) Inventors: Xinyi Cynthia Chen, Calabasas, CA (US); Shalini Gupta, Newbury Park, CA (US); Francesca Civoli, Newbury Park, CA (US)

(73) Assignee: AMGEN INC., Thousand Oaks, CA (US)

(21) Appl. No.: 13/816,442

(22) PCT Filed: Aug. 10, 2011
(86) PCT No.: PCT/US11/47289

§ 371 Date: Oct. 28, 2013

Related U.S. Application Data
(60) Provisional application No. 61/372,432, filed on Aug. 10, 2010.

(51) Int. Cl. G01N 33/577

(52) U.S. Cl. CPC G01N 33/577
USPC 435/7.21; 436/501; 435/7.5

(57) ABSTRACT

An in vitro assay method is disclosed. This non-cell-based dual function target binding assay is useful for detecting both an IgG target antibody, such as a biologic drug, in a biological sample (e.g., a serum sample) and the presence of neutralizing antibodies (NAb) against the IgG target antibody.

The instant application contains an ASCII “txt” compliant sequence listing submitted via EFS-WEB on Aug. 10, 2010, which serves as both the computer readable form (CRF) and the paper copy required by 37 C.F.R. Section 1.821(c) and 1.821(e), and is hereby incorporated by reference in its entirety. The name of the “txt” file created on Aug. 10, 2010, is: A-1586-US-PSP-SeqList081010_ST25.txt, and is 25 kb in size.

Throughout this application various publications are referenced within parentheses or brackets. The disclosures of these publications in their entireties are hereby incorporated by reference in this application in order to more fully describe the state of the art to which this invention pertains.

### BACKGROUND OF THE INVENTION

1. Field of the Invention

The present invention relates to the field of therapeutic antibodies.

2. Discussion of the Related Art

Antibody-Dependent Cell-Mediated Cytotoxicity (ADCC) is one of the mechanisms of humoral immune response. In an ADCC response, an effector cell of the immune system, typically a natural killer (NK) cell, actively lyses a target cell that has been bound by specific antibodies that have bound to a target protein on the surface of the target cell. Neutrophils and eosinophils can also mediate ADCC. For example, eosinophils can kill certain parasitic worms known as helminths through ADCC.

Antibodies of various IgG isotypes have been reported to have some ADCC activity, but typically, IgG1 and IgG3 isotypes are characterized as having significant antibody-dependent cellular cytotoxicity (ADCC) activity. In one study, both IgG1 and IgG3 antibodies were reported to be equally effective in mediating monocyte or activated U937 cell ADCC; IgG1 was more active than IgG3 in NK-cell mediated ADCC. (Rozsnyay et al., Distinctive role of IgG1 and IgG3 isotypes in Fc gamma R-mediated functions, Immunology 66(4): 491-498 (1989)). IgG3-sensitized erythrocytes reportedly inhibited IgG1-induced lysis, implying that each subclass engages the same Fc gamma R receptor but that lysis requires a further ‘signal’ that the IgG3 molecule cannot deliver. (Rozsnyay et al., ibid.). FcγRIIIa (CD16a) has been identified as the relevant receptor for ADCC effector function.

Studies have revealed that fucose removal from the oligosaccharides of human IgG1 antibodies results in a significant enhancement of antibody-dependent cellular cytotoxicity (ADCC) via improved IgG1 binding to FcγRIIIa, and can reduce the antigen density required for ADCC induction via efficient recruitment and activation of NK cells. (Niwa et al., Enhanced Natural Killer Cell Binding and Activation by Low-Fucose IgG1 Antibody Results in Potent Antibody-Dependent Cellular Cytotoxicity Induction at Lower Antigen Density, Clinical Cancer Research 11: 2327 (2005); Satoh et al., Non-fucosylated therapeutic antibodies as next-generation therapeutic antibodies, Expert Opinion on Biological Therapy 6(11):1161-1173 (2006)). This phenomenon can be particularly useful in a therapeutic antibody. Therapeutic monoclonal antibodies, such as rituximab (Rituxan®) and trastuzumab (Herceptin®) are widely used in the treatment of neoplasms and/or autoimmune diseases. (Stavenhagen et al., Fc Optimization of Therapeutic Antibodies Enhances Their Ability to Kill Tumor Cells In vitro and Controls Tumor Expansion In vivo via Low-Affinity Activating Fcγ Receptors, Cancer Research 67(18):8882-90 (2007); Clynes, R A et al., Inhibitory Fc receptors modulate in vivo cytoxicity against tumor targets, Nat Med 6 (4): 443-46 (2000); Hauser et al., B-cell depletion with rituximab in relapsing-remitting multiple sclerosis, NEJM 358:676-88 (2008)).

KW-0761 (also known as “mogamulizumab” or “AMG 761”) is being developed for the treatment of patients with cutaneous T-cell lymphoma (CTCL) or peripheral T-cell lymphoma (PTCL). KW-0761 is a humanized monoclonal antibody of the immunoglobulin G, subclass 1 (IgG1) kappa isotype that targets CC chemokine receptor 4 (CCR4) expressing cells and has shown an ability to deplete T-lymphocytes expressing CCR4 via ADCC. KW-0761 has enhanced ADCC activity due to defucosylation from the complex-type oligosaccharide at the constant (Fc) region. (Ishii et al., Defucosylated Humanized Anti-CCR4Monoclonal Antibody KW-0761 as a Novel Immunotherapeutic Agent for Adult T-cell Leukemia/Lymphoma, Clinical Cancer Research 16: 1520-31 (2010); Shitara et al., Human CDR-grafted antibody and antibody fragment thereof, U.S. Pat. No. 7,504,104).

A neutralizing antibody, or NAb, is an immunoglobulin molecule that reacts with a specific antigen, which typically induced its in vivo synthesis, and with similar molecules. Neutralizing antibodies are classified according to mode of action as agglutinin, bacteriolysin, hemolysin, opsonin, or precipitin. Antibodies are synthesized by B lymphocytes that have been activated by the binding of an antigen to a cell-surface receptor, and neutralizing antibodies are able to eliminate or “neutralize” the biological effect of the antigen, which may be, for example on the surface of a pathogen. Unfortunately, therapeutic antibodies can also sometimes induce the production of NAbs in some patients, and it is important for the sake of clinical safety to be able to monitor the appearance of NAbs directed against the therapeutic antibody in the serum of a patient receiving a therapeutic antibody drug.

A reliable non-cell-based, in vitro assay method for detecting such therapeutic IgG antibodies that can induce ADCC, and also for detecting NAbs against such therapeutic antibodies in the serum of a patient are desired benefits that the present invention provides.

### SUMMARY OF THE INVENTION

The present invention is directed to an in vitro dual function target binding assay, which is useful for detecting both an IgG target antibody, such as a biologic drug, in a biological sample (e.g., a serum sample) and the presence of neutralizing antibodies (NAb) against the IgG target antibody.

In one embodiment, the inventive in vitro assay method comprises detecting in an avidin-coated well, by measuring a signal, in the presence of a fresh volume of a buffer permitting detection under physiological conditions, any of an antibody that specifically binds polyhistidine, that has bound a polyhistidine-tagged recombinant human CD16a polypeptide, wherein the avidin-coated well was previously blocked and subsequently a pre-incubated reaction mixture has been incubated in the blocked avidin-coated well, under physiological conditions, wherein the pre-incubated reaction mixture was suspended during its pre-incubation in an aqueous serum-containing assay buffer, and the pre-incubated reaction mixture comprised:

(i) a target antigen binding protein comprising an Fc domain with a CD16a binding site; and

(ii) a biotinylated polypeptide portion of a target protein of interest, to which polypeptide portion the target antigen binding protein specifically binds; and

wherein, subsequent to the incubation of the pre-incubated reaction mixture in the avidin-coated well, a polyhistidine-tagged recombinant human CD16a polypeptide suspended in a fresh volume of the assay buffer was incubated under physiological conditions, together with any target antigen binding protein that was bound to the biotinylated polypeptide portion that was bound to the avidin of the avidin-coated well; and wherein, prior to detecting by measuring the signal, the antibody that specifically binds polyhistidine, suspended in a fresh volume of the assay buffer, was incubated in the well under physiological conditions, together with any polyhistidine-tagged recombinant human CD16a polypeptide that was bound to the target antigen binding protein.

Some embodiments of the method further include, before detecting the antibody that specifically binds polyhistidine, the step of incubating in the well, under physiological conditions, an antibody that comprises a conjugated signal-producing label, suspended in a fresh volume of the assay buffer, wherein the antibody specifically binds the antibody that specifically binds polyhistidine.

In a particular embodiment, the inventive in vitro assay method involves the steps of

(a) incubating in a blocked avidin-coated well, under physiological conditions, a pre-incubated reaction mixture suspended in an aqueous serum-containing assay buffer, the pre-incubated reaction mixture comprising:

(i) an IgG target antibody comprising an Fc domain with a CD16a binding site; and

(ii) a biotinylated polypeptide portion of a target protein of interest, to which polypeptide portion the IgG target antibody specifically binds;

(b) incubating in the well, under physiological conditions, a polyhistidine-tagged recombinant human CD16a polypeptide suspended in a fresh volume of the assay buffer, together with any IgG target antibody that was bound to the biotinylated polypeptide portion that was bound to the avidin in (a);

(c) incubating in the well, under physiological conditions, an antibody that specifically binds polyhistidine, said antibody suspended in a fresh volume of the assay buffer, together with any polyhistidine-tagged recombinant human CD16a polypeptide that was bound to the IgG target antibody in (b); and

(d) detecting in the well, in the presence of a fresh volume of a buffer permitting detection under physiological conditions, any of the antibody that specifically binds polyhistidine that was bound to the polyhistidine-tagged recombinant human CD16a polypeptide in (c) by measuring a signal.

In some embodiments of the invention, the method further comprises, before step (d) the step of incubating in the well, under physiological conditions, an antibody that comprises a conjugated signal-producing label, suspended in a fresh volume of the assay buffer, wherein said antibody specifically binds the antibody that specifically binds polyhistidine in (c).

In another useful embodiment, the in vitro assay method involves the steps of:

(a) incubating in a blocked avidin-coated well, under physiological conditions, a pre-incubated reaction mixture suspended in an aqueous serum-containing assay buffer, the pre-incubated reaction mixture comprising:
(i) an IgG target antibody against human CCR4 comprising an Fc domain with a CD16a binding site;
(ii) serum sample to be tested for the presence of neutralizing antibodies; and
(iii) a biotinylated polypeptide portion of human CCR4, to which polypeptide portion the IgG target antibody specifically binds;

(b) incubating in the well, under physiological conditions, a polyhistidine-tagged recombinant human CD16a polypeptide suspended in a fresh volume of the assay buffer, together with any IgG target antibody that was bound to the biotinylated polypeptide portion of human CCR4 that was bound to the avidin in (a);

(c) incubating in the well, under physiological conditions, an antibody that specifically binds polyhistidine, said antibody suspended in a fresh volume of the assay buffer, together with any polyhistidine-tagged recombinant human CD 16a polypeptide that was bound to the IgG target antibody in (b);

(d) incubating in the well, under physiological conditions, an antibody that comprises a conjugated signal-producing label, suspended in a fresh volume of the assay buffer, wherein said antibody specifically binds the antibody that specifically binds polyhistidine in (c); and

(e) detecting in the well, in the presence of a fresh volume of a buffer permitting detection under physiological conditions, any signal produced.

In the various embodiments of the invention, the signal can be produced by a sensitive electrochemiluminescence (ECL) labeling system, but fluorescent label (e.g., fluorescein, phycoerythrin, phycocyanin, allophycocyanin, green fluorescent protein [GFP], enhanced GFP [eGFP], yellow fluorescent protein [YFP], cyan fluorescent protein [CFP], etc.), isotopic label (e.g., ¹²⁵I, ¹⁴C, ¹³C, ³⁵S, ³H, ²H, ¹³N, ¹⁵N, ¹⁸O, ¹⁷O, etc.), or enzyme-linked (e.g., horseradish peroxidase-, beta-galactosidase-, or luciferase-based) labeling systems are also useful embodiments. Signal is detected using a suitable instrument.

If the biological sample contains neutralizing antibodies against the IgG1 target antibody, the target antibody will not be able to bind to the biotinylated target peptide of interest that is captured on the avidin-coated solid substrate. Therefore, a low (e.g., ECL) signal is generated in the presence of NAb. In the absence of NAb, a high (e.g., ECL) signal is generated because the IgG1 target antibody is able be build a bridge between the biotinylated target peptide and the polyhistidine-tagged recombinant human FCγRIIIa (CD16a).

Numerous additional aspects and advantages of the present invention will become apparent upon consideration of the figures and detailed description of the invention.

### BRIEF DESCRIPTION OF THE DRAWINGS

FIG. 1 shows a schematic representation of an embodiment of the inventive dual function assay, as configured for detecting an IgG1 target antibody (e.g., KW-0761, also known as “mogamulizumab” or “AMG 761”) that specifically binds a biotinylated (“B”) target protein of interest (e.g., CCR4), and for detecting neutralizing antibodies in a serum sample. In this embodiment, the avidin-coated plate is represented by “MSD 6000 plate” (Meso Scale Discovery, Gaithersburg, Md.) coated with streptavidin (“SA”), and a SULFO-TAG™ protein conjugate (Meso Scale Discovery, Gaithersburg, Md.) electrochemiluminescence (ECL) antibody labeling system is employed for detection purposes. A SECTOR® Imager 6000 plate reader (“MSD 6000”) was employed in detection.

FIG. 2 shows a flowchart of steps of an embodiment of the inventive in vitro assay for detecting neutralizing antibodies against an IgG1 target antibody.

FIG. 3 shows a representative dose response for KW-0761 (also known as “mogamulizumab” or “AMG 761”) in the inventive assay in the presence of assay buffer (0% PHS) or pooled human serum (“PHS”: 5% PHS or 20% PHS; (v/v)).

FIG. 4 demonstrates that binding of KW-0761 (also known as “mogamulizumab” or “AMG 761”) to the biotinylated CCR4 peptide is inhibited by the presence of polyclonal anti-KW-0761 neutralizing antibodies in a dose dependent manner.

FIG. 5 demonstrates that rituximab binds to biotinylated CD20 peptides in a dose dependent manner in the same assay format represented schematically in FIG. 1 and FIG. 2, as disclosed and modified in Example 3. “MBT4736” refers to SEQ ID NO:7; “MBT4737” refers to SEQ ID NO:8.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, fusion proteins can be derivatized as described herein by well-known organic chemistry techniques.

The term “isolated protein” referred means that a subject protein (1) is free of at least some other proteins with which it would normally be found in nature, (2) is essentially free of other proteins from the same source, e.g., from the same species, (3) is expressed recombinantly by a cell of a heterologous species or kind, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, and/or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof may encode such an isolated protein. Preferably, the isolated protein is substantially free from proteins or polypeptides or other contaminants that are found in its natural environment that would interfere with its therapeutic, diagnostic, prophylactic, research or other use.

In further describing any of the polypeptides or proteins herein, as well as variants, a one-letter abbreviation system is frequently applied to designate the identities of the twenty “canonical” amino acid residues generally incorporated into naturally occurring peptides and proteins (Table 1). Such one-letter abbreviations are entirely interchangeable in meaning with three-letter abbreviations, or non-abbreviated amino acid names.

TABLE 1
One-letter abbreviations for the canonical amino acids.
Three-letter abbreviations are in parentheses.

| | |
|---|---|
| Alanine (Ala) | A |
| Glutamine (Gln) | Q |
| Leucine (Leu) | L |
| Serine (Ser) | S |
| Arginine (Arg) | R |
| Glutamic Acid (Glu) | E |
| Lysine (Lys) | K |
| Threonine (Thr) | T |
| Asparagine (Asn) | N |
| Glycine (Gly) | G |
| Methionine (Met) | M |
| Tryptophan (Trp) | W |
| Aspartic Acid (Asp) | D |
| Histidine (His) | H |
| Phenylalanine (Phe) | F |
| Tyrosine (Tyr) | Y |
| Cysteine (Cys) | C |
| Isoleucine (Ile) | I |
| Proline (Pro) | P |
| Valine (Val) | V |

An amino acid substitution in an amino acid sequence is typically designated herein with a one-letter abbreviation for the amino acid residue in a particular position, followed by the numerical amino acid position relative to an original sequence of interest, which is then followed by the one-letter symbol for the amino acid residue substituted in. For example, “T30D” symbolizes a substitution of a threonine residue by an aspartate residue at amino acid position 30, relative to the original sequence of interest. Another example, “W101F” symbolizes a substitution of a tryptophan residue by a phenylalanine residue at amino acid position 101, relative to the original sequence of interest. Non-canonical amino acid residues can be incorporated into a peptide within the scope of the invention by employing known techniques of protein engineering that use recombinantly expressing cells. (See, e.g., Link et al., Non-canonical amino acids in protein engineering, Current Opinion in Biotechnology, 14(6):603-609 (2003)). The term “non-canonical amino acid residue” refers to amino acid residues in D- or L-form that are not among the 20 canonical amino acids generally incorporated into naturally occurring proteins, for example, β-amino acids, homoamino acids, cyclic amino acids and amino acids with derivatized side chains. Examples include (in the L-form or D-form) β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine, isodesmosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyllysine, O-phosphoserine, N^{α}-acetylserine, N^{α}-formylmethionine, 3-methylhistidine, 5-hydroxylysine, and other similar amino acids, and those listed in Table 2 below, and derivatized forms of any of these as described herein. Table 2 contains some exemplary non-canonical amino acid residues that are useful in accordance with the present invention and associated abbreviations as typically used herein, although the skilled practitioner will understand that different abbreviations and nomenclatures may be applicable to the same substance and appear interchangeably herein.

TABLE 2
Useful non-canonical amino acids for amino acid addition,
insertion, or substitution into peptide sequences in accordance
with the present invention. In the event an abbreviation listed
in Table 2 differs from another abbreviation for the same
substance disclosed elsewhere herein, both abbreviations are
understood to be applicable. The amino acids listed in Table 2
can be in the L-form or D-form.

| Amino Acid | Abbreviation(s) |
|---|---|
| Acetamidomethyl | Acm |
| Acetylarginine | acetylarg |
| α-aminoadipic acid | Aad |
| aminobutyric acid | Abu |
| 6-aminohexanoic acid | Ahx; εAhx |
| 3-amino-6-hydroxy-2-piperidone | Ahp |
| 2-aminoindane-2-carboxylic acid | Aic |
| α-amino-isobutyric acid | Aib |
| 3-amino-2-naphthoic acid | Anc |
| 2-aminotetraline-2-carboxylic acid | Atc |
| aminophenylalanine | Aminophe; Amino-Phe |
| 4-amino-phenylalanine | 4AmP |
| 4-amidino-phenylalanine | 4AmPhe |
| 2-amino-2-(1-carbamimidoylpiperidin-4- | 4AmPig |
| yl)acetic acid | |
| Arg ψ(CH₂NH)-reduced amide bond | rArg |
| β-homoarginine | bhArg |
| β-homolysine | bhomoK |
| β-homo Tic | BhTic |
| β-homophenylalanine | BhPhe |
| β-homoproline | BhPro |
| β-homotryptophan | BhTrp |
| 4,4′-biphenylalanine | Bip |
| β,β-diphenyl-alanine | BiPhA |
| β-phenylalanine | BPhe |
| p-carboxyl-phenylalanine | Cpa |
| citrulline | Cit |
| cyclohexylalanine | Cha |
| cyclohexylglycine | Chg |
| cyclopentylglycine | Cpg |
| 2-amino-3-guanidinopropanoic acid | 3G-Dpr |
| α,γ-diaminobutyric acid | Dab |
| 2,4-diaminobutyric acid | Dbu |
| diaminopropionic acid | Dap |
| α,β-diaminopropionoic acid (or 2,3- | Dpr |
| diaminopropionic acid | |
| 3,3-diphenylalanine | Dip |
| 4-guanidino phenylalanine | Guf |
| 4-guanidino proline | 4GuaPr |
| homoarginine | hArg; hR |
| homocitrulline | hCit |
| homoglutamine | hQ |
| homolysine | hLys; hK; homoLys |
| homophenylalanine | hPhe; homoPhe |
| 4-hydroxyproline (or hydroxyproline) | Hyp |
| 2-indanylglycine (or indanylglycine) | IgI |
| indoline-2-carboxylic acid | Idc |
| Iodotyrosine | I-Tyr |
| Lys ψ(CH₂NH)-reduced amide bond | rLys |
| methionine oxide | Met[O] |
| methionine sulfone | Met[O]₂ |
| N^{α}-methylarginine | NMeR |
| Nα-[(CH₂)₃NHCH(NH)NH₂] substituted | N-Arg |
| glycine | |
| N^{α}-methylcitrulline | NMeCit |
| N^{α}-methylglutamine | NMeQ |
| N^{α}-methylhomocitrulline | N^{α}-MeHoCit |
| N^{α}-methylhomolysine | NMeHoK |
| N^{α}-methylleucine | N^{α}-MeL; NMeL; NMeLeu; |
| | NMe-Leu |
| N^{α}-methyllysine | NMe-Lys |
| Nε-methyl-lysine | N-eMe-K |
| Nε-ethyl-lysine | N-eEt-K |
| Nε-isopropyl-lysine | N-eIPr-K |
| N^{α}-methylnorleucine | NMeNle; NMe-Nle |
| N^{α}-methylornithine | N^{α}-MeOrn; NMeOrn |
| N^{α}-methylphenylalanine | NMe-Phe |
| 4-methyl-phenylalanine | MePhe |
| α-methylphenyalanine | AMeF |
| N^{α}-methylthreonine | NMe-Thr; NMeThr |
| N^{α}-methylvaline | NMeVal; NMe-Val |
| Nε-(O-(aminoethyl)-O′-(2-propanoyl)- | K(NPeg11) |
| undecaethyleneglycol)-Lysine | |
| Nε-(O-(aminoethyl)-O′-(2-propanoyl)- | K(NPeg27) |
| (ethyleneglycol)27-Lysine | |
| 3-(1-naphthyl)alanine | 1-Nal; 1Nal |
| 3-(2-naphthyl)alanine | 2-Nal; 2Nal |
| nipecotic acid | Nip |
| nitrophenylalanine | nitrophe |
| norleucine | Nle |
| norvaline | Nva or Nvl |
| O-methyltyrosine | Ome-Tyr |
| octahydroindole-2-carboxylic acid | Oic |
| ornithine | Orn |
| Orn ψ(CH2NH)-reduced amide bond | rOrn |
| 4-piperidinylalanine | 4PipA |
| 4-pyridinylalanine | 4Pal |
| 3-pyridinylalanine | 3Pal |
| 2-pyridinylalanine | 2Pal |
| para-aminophenylalanine | 4AmP; 4-Amino-Phe |
| para-iodophenylalanine (or 4- | pI-Phe |
| iodophenylalanine) | |
| phenylglycine | Phg |
| 4-phenyl-phenylalanine (or | 4Bip |
| biphenylalanine) | |
| 4,4′-biphenyl alanine | Bip |
| pipecolic acid | Pip |
| 4-amino-1-piperidine-4-carboxylic acid | 4Pip |
| sarcosine | Sar |
| 1,2,3,4-tetrahydroisoquinoline | Tic |
| 1,2,3,4-tetrahydroisoquinoline-1- | Tiq |
| carboxylic acid | |
| 1,2,3,4-tetrahydroisoquinoline-7- | Hydroxyl-Tic |
| hydroxy-3-carboxylic acid | |
| 1,2,3,4-tetrahydronorharman-3- | Tpi |
| carboxylic acid | |
| thiazolidine-4-carboxylic acid | Thz |
| 3-thienylalanine | Thi |

Nomenclature and Symbolism for Amino Acids and Peptides by the UPAC-IUB Joint Commission on Biochemical Nomenclature (JCBN) have been published in the following documents: Biochem. J., 1984, 219, 345-373; Eur. J. Biochem., 1984, 138, 9-37; 1985, 152, 1; 1993, 213, 2; Internat. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; Biochemical Nomenclature and Related Documents, 2nd edition, Portland Press, 1992, pages 39-69.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

The term “fusion protein” indicates that the protein is a chimera including polypeptide components derived from more than one parental protein or polypeptide, or from the same protein but positioned within a single fusion molecule in a different order that is not naturally found together in the same protein molecule. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

A “secreted” protein refers to those proteins capable of being directed to the ER, secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments of the inventive composition, the polypeptide can be synthesized by the host cell as a secreted protein, which can then be further purified from the extracellular space and/or medium.

In several steps of the inventive in vitro assay method, a molecule or a mixture of molecules is “suspended” in an aqueous liquid, e.g., in a serum-containing assay buffer, or in a fresh volume of the assay buffer. The term “suspended” means that the molecule or mixture is dissolved in, interspersed in, floating within, moving within, or mixed in the liquid.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion, or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing a detergent or chaotropic agent, such as urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing a detergent or chaotropic agent, such as urea, guanidinium hydrochloride, or lithium perchlorate.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including an isotopic label (e.g., ¹²⁵I, ¹⁴C, ¹³C, ³⁵S, ³H, ²H, ¹³N, ¹⁵N, ¹⁸O, ¹⁷O, etc.), for ease of quantification or detection, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express a polypeptide (e.g., an oligopeptide or antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a polypeptide with portions from different proteins that are not naturally found together, or not found naturally together in the same sequence as present in the encoded fusion protein (i.e., a chimeric protein). Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. No. 6,022,952 and U.S. Pat. No. 6,335,178; Uemura et al., Protein expression vector and utilization thereof, U.S. Pat. No. 7,029,909; Ruben et al., 27 human secreted proteins, US 2003/0104400 A1).

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK-293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

“Mammal” for purposes of treatment refers to any animal classified as a mammal, including humans, domestic and farm animals, and zoo, sports, or pet animals, such as dogs, horses, cats, cows, rats, mice, monkeys, etc.

The term “naturally occurring” as used throughout the specification in connection with biological materials such as polypeptides, nucleic acids, host cells, and the like, refers to materials which are found in nature.

The term “antibody”, or interchangeably “Ab”, is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)₂, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity. Thus in some embodiments of the inventive in vitro assay method, the IgG target antibody is an IgG1 (e.g., KW-0761 (also known as “mogamulizumab” or “AMG 761”), rituximab, or trastuzumab) or IgG3 antibody.

The term “antigen binding protein” (ABP) includes antibodies or antibody fragments, as defined above, and recombinant peptides or other compounds that contain sequences derived from CDRs having the desired antigen-binding properties.

Antibody-antigen interactions can be characterized by the association rate constant in M⁻¹ s⁻¹ (kₐ), or the dissociation rate constant in s⁻¹ (k_{d}), or alternatively the dissociation equilibrium constant in M (K_{D}). In general, an antigen binding protein, such as an antibody or antibody fragment, “specifically binds” to an antigen when it has a significantly higher binding affinity for, and consequently is capable of distinguishing, that antigen, compared to its affinity for other unrelated proteins, under similar binding assay conditions. Desirable are characteristics such as binding affinity as measured by K_{D} (dissociation equilibrium constant) in the range of 10⁻⁹ M or lower, ranging down to 10⁻¹² M or lower (lower values indicating higher binding affinity), or avidity as measured by k_{d} (dissociation rate constant) in the range of 10⁻⁴ s⁻¹ or lower, or ranging down to 10⁻¹⁰ s⁻¹ or lower. Typically, an antigen binding protein (e.g., an antibody or antibody fragment) is said to “specifically bind” its target antigen when the dissociation equilibrium constant (K_{D}) is ≦10⁻⁸ M. The antigen binding protein (e.g., antibody or antibody fragment) specifically binds antigen with “high affinity” when the K_{D} is ≦5×10⁻⁹ M, and with “very high affinity” when the K_{D} is ≦5×10⁻¹° M. In one embodiment, the antigen binding protein (e.g., antibodies) will bind to with a K_{D} of between about 10⁻⁸ M and 10⁻¹⁰ M, and in yet another embodiment the antibodies will bind with a K_{D}≦5×10⁻⁹M. Association rate constants, dissociation rate constants, or dissociation equilibrium constants may be readily determined using kinetic analysis techniques such as surface plasmon resonance (BIAcore®; e.g., Fischer et al., A peptide-immunoglobulin-conjugate, WO 2007/045463 A1, Example 10, which is incorporated herein by reference in its entirety), or KinExA using general procedures outlined by the manufacturer or other methods known in the art. The kinetic data obtained by BIAcore® or KinExA may be analyzed by methods described by the manufacturer.

“Antigen binding region” or “antigen binding site” means a portion of an antigen binding protein (e.g., antibody protein or antibody fragment), that specifically binds a specified antigen. For example, that portion of an antibody that contains the amino acid residues that interact with an antigen and confer on the antibody its specificity and affinity for the antigen is referred to as “antigen binding region.” An antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen.

An “isolated” antibody is one that has been identified and separated from one or more components of its natural environment or of a culture medium in which it has been secreted by a producing cell. “Contaminant” components of its natural environment or medium are materials that would interfere with diagnostic or therapeutic uses for the antibody, and may include enzymes, hormones, and other proteinaceous or nonproteinaceous solutes. In some embodiments, the antibody will be purified (1) to greater than 95% by weight of antibody, and most preferably more than 99% by weight, or (2) to homogeneity by SDS-PAGE under reducing or nonreducing conditions, optionally using a stain, e.g., Coomassie blue or silver stain. Isolated naturally occurring antibody includes the antibody in situ within recombinant cells since at least one component of the antibody's natural environment will not be present. Typically, however, isolated antibody will be prepared by at least one purification step.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab′)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624-628[1991] and Marks et al., J. Mol. Biol., 222:581-597 (1991), for example.

The term “immunoglobulin” encompasses full antibodies comprising two dimerized heavy chains (HC), each covalently linked to a light chain (LC); a single undimerized immunoglobulin heavy chain and covalently linked light chain (HC+LC), or a chimeric immunoglobulin (light chain+heavy chain)-Fc heterotrimer (a so-called “hemibody”).

An “antibody” is a tetrameric glycoprotein. In a naturally-occurring antibody, each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa (κ) and lambda (λ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). Within the scope of the invention, an “antibody” also encompasses a recombinantly made antibody, and antibodies that are lacking glycosylation.

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains are classified as mu (μ), delta (Δ), gamma (γ), alpha (α), and epsilon (ε), and define the antibody's isotype as IgM, IgD, IgG, IgA, and IgE, respectively. In separate embodiments of the invention, heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface.

An “Fc region”, or used interchangeably herein, “Fc domain” or “immunoglobulin Fc domain”, contains two heavy chain fragments, which in a full antibody comprise the C_{H}1 and C_{H}2 domains of the antibody. The two heavy chain fragments are held together by two or more disulfide bonds and by hydrophobic interactions of the C_{H}3 domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

“Allotypes” are variations in antibody sequence, often in the constant region, that can be immunogenic and are encoded by specific alleles in humans. Allotypes have been identified for five of the human IGHC genes, the IGHG1, IGHG2, IGHG3, IGHA2 and IGHE genes, and are designated as G1m, G2m, G3m, A2m, and Em allotypes, respectively. At least 18 Gm allotypes are known: nG1m(1), nG1m(2), G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5). There are two A2m allotypes A2m(1) and A2m(2).

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5^{th} Ed. Public Health Service, National Institutes of Health, Bethesda, Md. (1991)]. Even a single CDR may recognize and bind antigen, although with a lower affinity than the entire antigen binding site containing all of the CDRs. In a typical antibody, the CDRs are embedded within a framework in the heavy and light chain variable region where they constitute the regions responsible for antigen binding and recognition. A variable region comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra).

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

“Antibody fragments” comprise a portion of an intact full length antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the VH and VL domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

A “Fab fragment” is comprised of one light chain and the C_{H}1 and variable regions of one heavy chain. The heavy chain of a Fab molecule cannot form a disulfide bond with another heavy chain molecule.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. No. 4,946,778 and No. 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Natl. Acad. Sci. USA 85:5879-5883, 1988). An “Fd” fragment consists of the V_{H} and C_{H}1 domains.

The term “diabodies” refers to small antibody fragments with two antigen-binding sites, which fragments comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “antigen” refers to a molecule or a portion of a molecule capable of being bound by a selective binding agent, such as an antigen binding protein (including, e.g., an antibody or immunologically functional fragment thereof), and additionally capable of being used in an animal to produce antibodies capable of binding to that antigen. An antigen may possess one or more epitopes that are capable of interacting with different antigen binding proteins, e.g., antibodies.

The term “epitope” is the portion of a molecule that is bound by an antigen binding protein (e.g., an antibody). The term includes any determinant capable of specifically binding to an antigen binding protein, e.g., an antibody. An epitope can be contiguous or non-contiguous (e.g., in a single-chain polypeptide, amino acid residues that are not contiguous to one another in the polypeptide sequence but that within the context of the molecule are bound by the antibody. In certain embodiments, epitopes may be mimetic in that they comprise a three dimensional structure that is similar to an epitope used to generate the antigen binding protein (e.g., an antibody), yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antibodies specific for a particular target antigen will preferentially recognize an epitope on the target antigen in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with polypeptides include, but are not limited to, one or more amino acid changes (including substitutions, insertions or deletions); chemical modifications; covalent modification by conjugation to therapeutic or diagnostic agents; labeling (e.g., with fluorescent label, electrochemiluminescent label, radionuclide or other isotopic label, or various enzymes); covalent polymer attachment such as PEGylation (derivatization with polyethylene glycol) and insertion or substitution by chemical synthesis of non-natural amino acids. Modified polypeptides should retain the binding properties of unmodified molecules used in the inventive method.

“Conjugated” means that at least two chemical moieties are covalently linked, or bound to each other, either directly, or optionally, via a peptidyl or non-peptidyl linker moiety that is itself covalently linked to both of the moieties. For example, covalent linkage can be via an amino acid residue of a peptide or protein, including via an alpha amino group, or via a side chain.

“Under physiological conditions” with respect to incubating buffers or reagents of the inventive assay method means incubation under conditions of temperature, pH, and ionic strength, that permit a biochemical reaction, such as a non-covalent binding reaction, to occur. Typically, the temperature is at room or ambient temperature up to about 37° C. and at pH 6.5-7.5.

“Blocked” means pre-incubated with assay buffer, e.g., for the purpose of minimizing non-specific binding to well surfaces by reaction mixture components when the reaction mixture is later added.

A “reaction mixture” is an aqueous mixture containing all the reagents and factors necessary, which under physiological conditions of incubation, permit an in vitro biochemical reaction of interest to occur, such as a non-covalent binding reaction.

“Avidin” is a tetrameric biotin-binding protein, naturally produced in the oviducts of birds, reptiles and amphibians and typically deposited in the whites of their eggs, or a synthetically or recombinantly produced version thereof, and/or a monomeric or multimeric (e.g., dimeric, trimeric, etc.) form thereof. In its tetrameric, glycosylated form, avidin is estimated to be between 66-69 kDa in size and can bind up to four molecules of biotin simultaneously with a high degree of affinity and specificity. (Korpela, J., Avidin, a high affinity biotin-binding protein as a tool and subject of biological research. Med. Bio. 62:5-26 (1984)). For purposes of the present invention, “avidin” can be glycosylated, deglycosylated, or non-glycosylated, derivatized, or modified, as long as the avidin maintains a high affinity for biotin (dissociation equilibrium constant K_{D} in the order of 10⁻¹⁴ M to 10⁻¹⁶ M). Included within the meaning of “avidin” are neutravidin (or NeutrAvidin) and streptavidin. “Streptavidin”, naturally produced by the bacterium Streptomyces avidinii as a 52,800 Da tetrameric biotin-binding protein, and includes a purified or synthetically or recombinantly produced version thereof, and/or a monomeric or multimeric (e.g., dimeric, trimeric, etc.) form thereof. Recombinantly engineered monovalent or multivalent forms of avidin (or streptavidin) are also included within “avidin”. (e.g., Laitinen et al. Genetically engineered avidins and streptavidins, Cell Mol Life Sci. 63 (24): 2992-30177 (2006); Howarth et al., A monovalent streptavidin with a single femtomolar biotin binding site, Nat Methods 3(4):267-73 (2006)). “Avidin-coated” means that a solid surface, e.g., a well-surface of a polystyrene plate, is conjugated with avidin moieties such that the avidin moieties are still able to bind biotin non-covalently with high affinity under physiological conditions, e.g., with a dissociation equilibrium constant K_{D} in the order of 10⁻¹⁴ M to 10⁻¹⁶ M. In some embodiments of the avidin-coated well in the inventive in vitro assay, the avidin employed is streptavidin or neutravidin.

A “well” is a chamber capable of receiving, through a coverable opening, and containing an aqueous liquid. For example, each individual compartment of a microtiter plate (e.g., 96- or 384-well microtiter plate) is a “well”; a single-compartment vessel, such as a culture plate, Petri dish, test tube, conical tube, or the depression of a depression slide is also a “well”. The openings of wells may be uncovered or covered separately by removable individual covers, or collectively by a single removable cover.

“Biotin” is a water-soluble B-complex vitamin, i.e., vitamin B7, that is composed of an ureido (tetrahydroimidizalone) ring fused with a tetrahydrothiophene ring (See, Formula I).

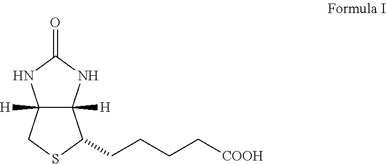 [SMILES: O=C(O)CCCC[C@@H]1SC[C@@H]2NC(=O)N[C@@H]21]

A valeric acid substituent is attached to one of the carbon atoms of the tetrahydrothiophene ring. In nature, biotin is a coenzyme in the metabolism of fatty acids and leucine, and it plays a role in vivo in gluconeogenesis. Biotin binds very tightly to the tetrameric protein avidin (e.g., Chicken avidin, bacterial streptavidin, and neutravidin), with a dissociation equilibrium constant K_{D} in the order of 10⁻¹⁴ M to 10⁻¹⁶ M, which is one of the strongest known protein-ligand interactions, approaching the covalent bond in strength. (Laitinen et al. Genetically engineered avidins and streptavidins, Cell Mol Life Sci. 63 (24): 2992-30177 (2006)). The biotin-avidin non-covalent interaction is often used in different biotechnological applications. (See, Laitinen et al., Genetically engineered avidins and streptavidins, Cell Mol Life Sci. 63 (24): 2992-30177 (2006)).

“Biotinylated” means that a substance is conjugated to one or more biotin moieties. Biotinylated peptides useful in practicing the invention can be purchased commercially (e.g., Midwest Bio-Tech Inc.) or can be readily synthesized and biotinylated. Biotinylation of compounds, such as peptides, can be by any known chemical technique. These include primary amine biotinylation, sulfhydryl biotinylation, and carboxyl biotinylation. For example, amine groups on the peptide, which are present as lysine side chain epsilon-amines and N-terminal α-amines, are common targets for primary amine biotinylation biotinylation. Amine-reactive biotinylation reagents can be divided into two groups based on water solubility.
1) N-hydroxysuccinimide (NHS)-esters of biotin have poor solubility in aqueous solutions. For reactions in aqueous solution, they must first be dissolved in an organic solvent, then diluted into the aqueous reaction mixture. The most commonly used organic solvents for this purpose are dimethyl sulfoxide (DMSO) and dimethyl formamide (DMF), which are compatible with most proteins at low concentrations.
2) Sulfo-NHS-esters of biotin are more soluble in water, and are dissolved in water just before use because they hydrolyze easily. The water solubility of sulfo-NHS-esters stems from their sulfonate group on the N-hydroxysuccinimide ring and eliminates the need to dissolve the reagent in an organic solvent.
   Chemical reactions of NHS- and sulfo-NHS-esters are essentially the same: an amide bond is formed and NHS or sulfo-NHS become leaving groups. Because the targets for the ester are deprotonated primary amines, the reaction is prevalent above pH 7. Hydrolysis of the NHS-ester is a major competing reaction, and the rate of hydrolysis increases with increasing pH. NHS- and sulfo-NHS-esters have a half-life of several hours at pH 7, but only a few minutes at pH 9. The conditions for conjugating NHS-esters to primary amines of peptides include incubation temperatures in the range 4-37° C., reaction pH values in the range 7-9, and incubation times from a few minutes to about 12 hours. Buffers containing amines (such as Tris or glycine) must be avoided because they compete with the reaction. The HABA dye (2-(4-hydroxyazobenzene)benzoic acid) method can be used to determine the extent of biotinylation. Briefly, HABA dye is bound to avidin and yields a characteristic absorbance. When biotin, in the form of biotinylated protein or other molecule, is introduced, it displaces the dye, resulting in a change in absorbance at 500 nm. The absorbance change is directly proportional to the level of biotin in the sample.

“FCγRIIIa”, also known as “CD16a” or “FCGR3” or “IGFR3”, which designations are used interchangeably herein, means immunoglobulin G Fc receptor III. Human CD16a (GenBank Accession AAH17865) comprises the amino acid sequence:

1 mwqlllptal lllvsagmrt edlpkavvfl epqwyrvlek dsvtlkcqga yspednstqw

61 fhneslissq assyfidaat vddsgeyrcq tnlstlsdpv qlevhigwll lqaprwvfke

121 edpihlrchs wkntalhkvt ylqngkgrky fhhnsdfyip katlkdsgsy fcrglvgskn

181 vssetvniti tqglaystis sffppgyqvs fclvmvllfa vdtglyfsvk tnirsstrdw

241 kdhkfkwrkd pqdk//SEQ ID NO:11.

However, polymorphic sequences of CD16a, are also encompassed by the term“CD16a”. Such polymorphisms include 176V (high binding form) and 176F (low binding form). The 254-amino acid residue sequence of CD16a (SEQ ID NO:11) includes a 16-residue signal sequence; a 191-residue extracellular domain (ECD); and a 22-residue transmembrane domain and a 25-residue C-terminal cytoplasmic domain. A “recombinant human CD16a polypeptide” is a fragment of SEQ ID NO:11 (or a polymorphic variant thereof) produced by recombinant DNA techniques that is soluble. An example of such a soluble fragment is the G17-Q208 fragment of SEQ ID NO:11, which comprises the putative ECD:

17 gmrt edlpkavvfl epqwyrvlek dsvtlkcqga yspednstqw

61 fhneslissq assyfidaat vddsgeyrcq tnlstlsdpv qlevhigwll lqaprwvfke

121 edpihlrchs wkntalhkvt ylqngkgrky fhhnsdfyip katlkdsgsy fcrglvgskn

181 vssetvniti tqglaystis sffppgyq//SEQ ID NO:12. Other embodiments of “recombinant human CD16a polypeptide” include smaller fragments of SEQ ID NO:12 that maintain the ability to bind human IgG with an estimated K_{D} less than 50 nM.

“Polyhistidine-tagged” means that the recombinant human CD16a polypeptide is conjugated, either by synthetic techniques (e.g., Peterson, U.S. Pat. No. 5,840,834, Technique for joining amino acid sequences and novel composition useful in immunoassays), or by recombinant DNA techniques, as a N-terminal or C-terminal extension of the recombinant human CD16a polypeptide comprising at least five, but typically, six (“hexa histidine-tag” or “6×His-tag”) to 18, contiguous histidine residues. Polyhistidine-tagged recombinant human CD16a polypeptide is also commercially available (e.g., R&D Systems Catalog #4325-FC). Antibodies that specifically bind polyhistidine and methods for making them are well known in the art, and such antibodies are commercially available. (e.g., Zentgraf et al., Antibodies active against a fusion polypeptide comprising a histidine portion, U.S. Pat. No. 6,790,940 and U.S. Pat. No. 7,713,712; Sigma-Aldrich Product #H1029; R&D Systems Catalog #MAB050). In some embodiments of the inventive in vitro assay method, the antibody that specifically binds polyhistidine, or, in other embodiments, the antibody that specifically binds the antibody that specifically binds polyhistidine, further comprises, conjugated to the antibody, a signal-producing label, such as but not limited to, a fluorescent label, an isotopic label, an electrochemiluminescent label, or an enzyme that can catalyze a reaction that turns a substrate into a reactant that is readily detectable by spectrophotometric, colorimetric, fluorometric, luminometric or other instruments (e.g., horseradish peroxidase-, beta-galactosidase-, or luciferase-based detection systems).

“Electrochemiluminescence” (ECL), or interchangeably “electrogenerated chemiluminescence”, is a kind of luminescence generated during electrochemical reactions in solutions. In ECL, electrochemically generated intermediates undergo a highly exergonic reaction to produce an electronically excited state that then emits light. (Forster et al., Electrogenerated Chemiluminescence, Annual Review of Analytical Chemistry 2: 359-385 (2009)). ECL excitation is caused by energetic electron transfer (redox) reactions of electrogenerated species. These electron-transfer reactions are sufficiently exergonic to allow the excited states of luminophores, including polycyclic aromatic hydrocarbons and metal complexes, to be created without photoexcitation. For example, oxidation of [Ru(bpy)₃]²⁺ in the presence of tripropylamine results in light emission that is analogous to the emission produced by photoexcitation. Such ruthenium complexes can usefully be employed as chemiluminescent labels for purposes of the invention. For example, the Meso Scale Discovery (MSD; Gaithersburg, Md.) Sulfo-Tag™ ECL detection system relies on a electrochemiluminescent label containing a ruthenium complex. In the Sulfo-Tag™ system, an activated N-hydroxysucccinimide ester having the following chemical structure (II) is used to form the label:

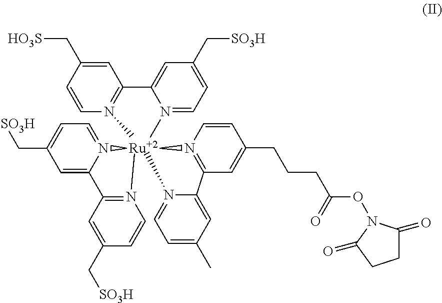

Following manufacturer's instructions (MSD Sulfo-Tag™ NHS-Ester, 17794-v2-2008 May, (2008)), the activated N-hydroxysucccinimide ester is reacted with a polypeptide to be labeled, such as an antibody. As previously mentioned, chemical reactions of NHS- and sulfo-NHS-esters are essentially the same: an amide bond is formed and NHS or sulfo-NHS become leaving groups. Because the targets for the ester are deprotonated primary amines, the reaction is prevalent above pH 7. The conditions for conjugating NHS-esters to primary amines of peptides include incubation temperatures in the range 4-37° C., reaction pH values in the range 7-9, and incubation times from a few minutes to about 12 hours. Buffers containing amines (such as Tris or glycine) must be avoided because they compete with the reaction. The labeled polypeptide product of the labeling reaction includes the electrochemiluminescent label comprising a ruthenium complex having the following formula (III), wherein the line drawn from the carbonyl group shows the attachment to the rest of the molecule:

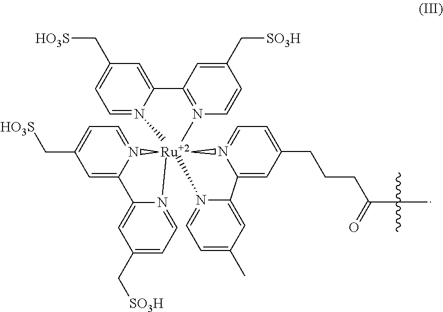

In some embodiments of the inventive in vitro assay method, the pre-incubated reaction mixture further contains a serum sample to be tested for the presence of neutralizing antibodies. “Neutralizing antibodies” (NAb) are antibodies that are capable of reducing the serum titer of an antigen of interest, for example, an IgG target antibody, by specifically binding to it, in vivo or in a sample in vitro. In vivo, NAbs block the biological activity of the therapeutic molecule by either binding directly to epitope(s) that lie within the active site of the therapeutic molecule or by blocking its active site by steric hindrance due to binding to epitope(s) that may lie in close proximity to the active site. While, in certain cases NAb presence may not result in a clinical effect, at sufficient NAb levels in other cases, a decrease in efficacy may be observed which may require administration of higher doses of the drug product in order to achieve similar efficacy. (See, e.g., Gupta et al., Recommendations for the design, optimization, and qualification of cell-based assays used for the detection of neutralizing antibody responses elicited to biological therapeutics, Journal of Immunological Methods 321(1-2):1-18 (2007).)

in other cases, a decrease in efficacy may be observed which may require administration of higher doses of the drug product in order to achieve similar efficacy.

“Target protein” of interest is any protein, such as but not limited to a human protein, that is of scientific, medical, clinical, or therapeutic interest as the specific target for an antibody. An example of a target protein of interest is chemokine receptor type 4 (CCR4). The amino acid sequence of human CCR4 is the following (Genbank Accession P51679):

| SEQ ID NO: 1 | |
|---|---|
| 1 | mnptdiadtt ldesiysnyy lyesipkpct kegikafgel flpplyslvf vfgllgnsvv |
| 61 | vlvlfkykrl rsmtdvylln laisdllfvf slpfwgyyaa dqwvfglglc kmiswmylvg |
| 121 | fysgiffvml msidrylaiv havfslrart ltygvitsla twsvavfasl pgflfstcyt |
| 181 | ernhtycktk yslnsttwkv lssleinilg lviplgimlf cysmiirtlq hcknekknka |
| 241 | vkmifavvvl flgfwtpyni vlfletivel evlqdctfer yldyaiqate tlafvhccln |
| 301 | piiyfflgek frkyilqlfk tcrglfvlcq ycgllqiysa dtpsssytqs tmdhdlhdal //. |

Another example of a target protein of interest is B-lymphocyte antigen CD20. The amino acid sequence of human CD20 is the following (Genbank Accession NP_{—}690605):

| SEQ ID NO: 2 | |
|---|---|
| 1 | mttprnsvng tfpaepmkgp iamqsgpkpl frrmsslvgp tqsffmresk tlgavqimng |
| 61 | lfhialggll mipagiyapi cvtvwyplwg gimyiisgsl laateknsrk clvkgkmimn |
| 121 | slslfaaisg milsimdiln ikishflkme slnfirahtp yiniyncepa npseknspst |
| 181 | qycysiqslf lgilsvmlif affqelviag ivenewkrtc srpksnivll saeekkeqti |
| 241 | keevvglt etssqpknee dieiipiqee eeeetetnfp eppqdqessp iendssp//. |

Another example of a target protein of interest is HER2. The amino acid sequence of human HER2 (also known as oncogene ERBB2, tyrosine-protein kinase erbB-2, oncogene NGL, NEU, or TKR1) is the following (Genbank Accession P04626):

| SEQ ID NO: 13 | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dpinnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktlspgkngv vkdvfafgga venpeyltpq |
| 1201 | ggaapqphpp pafspafdnl yywdqdpper gappstfkgt ptaenpeylg ldvpv//. |

A “polypeptide portion” is a subset of a target protein of interest that comprises an antigenic peptidyl portion of the target protein. Typically, such a “polypeptide portion” comprises at least 5 to 6 contiguous amino acid residues and can be as large as the entire protein of interest. More typically, the “polypeptide portion” is 10 to 40 amino acid residues in length. The “polypeptide portion” includes the amino acid residues of the target protein to which at least one CDR, or in another embodiment at least two CDRs, or in still another embodiment at least three CDRs, of the target antigen binding protein or IgG target antibody specifically binds.

“Target antigen binding protein” is an antigen binding protein that specifically binds to the polypeptide portion of the target protein of interest, and also includes an Fc domain with a CD 16a binding site. An IgG target antibody is an example of a “target antigen binding protein.”

“IgG target antibody” is an antibody that specifically binds to the polypeptide portion of the target protein of interest, and also includes an Fc domain with a CD 16a binding site.

Production of Antibodies

Polyclonal Antibodies.

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies.

Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. For example, monoclonal antibodies may be made using the hybridoma method first described by Kohler et al., Nature, 256:495 (1975), or may be made by recombinant DNA methods (e.g., Cabilly et al., Methods of producing immunoglobulins, vectors and transformed host cells for use therein, U.S. Pat. No. 6,331,415), including methods, such as the “split DHFR” method, that facilitate the generally equimolar production of light and heavy chains, optionally using mammalian cell lines (e.g., CHO cells) that can glycosylate the antibody (See, e.g., Page, Antibody production, EP0481790 A2 and U.S. Pat. No. 5,545,403).

Monoclonal

In the hybridoma method, a mouse or other appropriate host mammal, such as rats, hamster or macaque monkey, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells, once prepared, are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by BIAcore® or Scatchard analysis (Munson et al., Anal. Biochem., 107:220 (1980); Fischer et al., A peptide-immunoglobulin-conjugate, WO 2007/045463 A1, Example 10, which is incorporated herein by reference in its entirety).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

Hybridomas or mAbs may be further screened to identify mAbs with particular properties, such as binding affinity with a particular antigen or target. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, affinity chromatography, or any other suitable purification technique known in the art.

Recombinant Production of Antibodies and Other Polypeptides.

The invention provides isolated nucleic acids encoding any of the polypeptides, fusion peptides, or antigen binding proteins, such as antibodies (polyclonal and monoclonal), and including antibody fragments, of the invention described herein, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium. Similar materials and methods apply to production of other polypeptides.

Relevant amino acid sequences from an immunoglobulin or polypeptide of interest may be determined by direct protein sequencing, and suitable encoding nucleotide sequences can be designed according to a universal codon table. Alternatively, genomic or cDNA encoding the monoclonal antibodies may be isolated and sequenced from cells producing such antibodies using conventional procedures (e.g., by using oligonucleotide probes that are capable of binding specifically to genes encoding the heavy and light chains of the monoclonal antibodies).

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

The sequence encoding an entire variable region of the immunoglobulin polypeptide may be determined; however, it will sometimes be adequate to sequence only a portion of a variable region, for example, the CDR-encoding portion. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, one of skill will readily be able to determine, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the antibody; e.g., ATGGACATGAGGGTGCCCGCTCAGCTCCTGGGGCTCCTGCTGCTGTGGCTGAGAGGT GCGCGCTGT//SEQ ID NO:9, which encodes the VK-1 signal peptide sequence MDMRVPAQLLGLLLLWLRGARC//SEQ ID NO:10), an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus such as B. subtilis and B. licheniformis, Pseudomonas, and Streptomyces. Eukaryotic microbes such as filamentous fungi or yeast are suitable cloning or expression hosts for recombinant polypeptides or antibodies. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluvveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of polypeptides (including antigen binding proteins, e.g., antibodies and antibody fragments) from such cells has become routine procedure. Examples of useful mammalian host cell lines are Chinese hamster ovary cells, including CHOK1 cells (ATCC CCL61), DXB-11, DG-44, and Chinese hamster ovary cells/−DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y. Acad. Sci. 383: 44-68 (1982)); MRC 5 cells or FS4 cells; or mammalian myeloma cells.

Host cells are transformed or transfected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The host cells used to produce the polypeptides useful in the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the recombinant polypeptide can be produced intracellularly, in the periplasmic space, or directly secreted into the medium. If the polypeptide, such as an antigen binding protein (e.g., an antibody), is produced intracellularly, as a first step, the particulate debris, either host cells or lysed fragments, is removed, for example, by centrifugation or ultrafiltration.

An antibody or antibody fragment can be purified using, for example, hydroxylapatite chromatography, cation or anion exchange chromatography, or preferably affinity chromatography, using the antigen of interest or protein A or protein G as an affinity ligand. Protein A can be used to purify proteins that include polypeptides are based on human γ1, γ2, or γ4 heavy chains (Lindmark et al., J. Immunol. Meth. 62: 1-13 (1983)). Protein G is recommended for all mouse isotypes and for human γ3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a C_{H}3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the antibody to be recovered.

Chimeric, Humanized, Human Engineered™, Xenomouse® Monoclonal Antibodies.

Chimeric monoclonal antibodies, in which the variable Ig domains of a rodent monoclonal antibody are fused to human constant Ig domains, can be generated using standard procedures known in the art (See Morrison, S. L., et al. (1984) Chimeric Human Antibody Molecules; Mouse Antigen Binding Domains with Human Constant Region Domains, Proc. Natl. Acad. Sci. USA 81, 6841-6855; and, Boulianne, G. L., et al, Nature 312, 643-646. (1984)). A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

Antibodies can also be produced using transgenic animals that have no endogenous immunoglobulin production and are engineered to contain human immunoglobulin loci. (See, e.g., Mendez et al., Nat. Genet. 15:146-156 (1997)) For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein. See also Jakobovits et al., Proc. Natl. Acad. Sci. USA, 90:2551 (1993); Jakobovits et al., Nature, 362:255-258 (1993); Bruggermann et al., Year in Immuno., 7:33 (1993); Mendez et al., Nat. Genet. 15:146-156 (1997); and U.S. Pat. No. 5,591,669, U.S. Pat. No. 5,589,369, U.S. Pat. No. 5,545,807; and U.S. Patent Application No. 20020199213. U.S. Patent Application No. and 20030092125 describes methods for biasing the immune response of an animal to the desired epitope. Human antibodies may also be generated by in vitro activated B cells (see U.S. Pat. Nos. 5,567,610 and 5,229,275).

Antibody Production by Phage Display Techniques

The development of technologies for making repertoires of recombinant human antibody genes, and the display of the encoded antibody fragments on the surface of filamentous bacteriophage, has provided another means for generating human-derived antibodies. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Typically, the Fd fragment (V_{H}-C_{H}1) and light chain (V_{L}-C_{L}) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning Antibody fragments specific for the antigen are enriched and finally isolated.

Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

A variety of procedures have been described for deriving human antibodies from phage-display libraries (See, for example, Hoogenboom et al., J. Mol. Biol., 227:381 (1991); Marks et al., J. Mol. Biol, 222:581-597 (1991); U.S. Pat. Nos. 5,565,332 and 5,573,905; Clackson, T., and Wells, J. A., TIBTECH 12, 173-184 (1994)). In particular, in vitro selection and evolution of antibodies derived from phage display libraries has become a powerful tool (See Burton, D. R., and Barbas III, C. F., Adv. Immunol. 57, 191-280 (1994); and, Winter, G., et al., Annu Rev. Immunol. 12, 433-455 (1994); U.S. patent application no. 20020004215 and WO92/01047; U.S. patent application no. 20030190317 published Oct. 9, 2003 and U.S. Pat. No. 6,054,287; U.S. Pat. No. 5,877,293. Watkins, “Screening of Phage-Expressed Antibody Libraries by Capture Lift,” Methods in Molecular Biology, Antibody Phage Display: Methods and Protocols 178: 187-193, and U.S. Patent Application Publication No. 20030044772 published Mar. 6, 2003 describes methods for screening phage-expressed antibody libraries or other binding molecules by capture lift, a method involving immobilization of the candidate binding molecules on a solid support.

Other Embodiments of Antigen Binding Proteins

As noted above, antibody fragments comprise a portion of an intact full length antibody, preferably an antigen binding or variable region of the intact antibody, and include linear antibodies and multispecific antibodies formed from antibody fragments. Nonlimiting examples of antibody fragments include Fab, Fab′, F(ab′)2, Fv, Fd, domain antibody (dAb), complementarity determining region (CDR) fragments, single-chain antibodies (scFv), single chain antibody fragments, maxibodies, diabodies, triabodies, tetrabodies, minibodies, linear antibodies, chelating recombinant antibodies, tribodies or bibodies, intrabodies, nanobodies, small modular immunopharmaceuticals (SMIPs), an antigen-binding-domain immunoglobulin fusion protein, a camelized antibody, a VHH containing antibody, or muteins or derivatives thereof, and polypeptides that contain at least a portion of an immunoglobulin that is sufficient to confer specific antigen binding to the polypeptide, such as a CDR sequence, as long as the antibody retains the desired biological activity. Such antigen fragments may be produced by the modification of whole antibodies or synthesized de novo using recombinant DNA technologies or peptide synthesis.

Additional antibody fragments include a domain antibody (dAb) fragment (Ward et al., Nature 341:544-546, 1989) which consists of a VH domain.

“Linear antibodies” comprise a pair of tandem Fd segments (VH-CH1-VH-CH1) which form a pair of antigen binding regions. Linear antibodies can be bispecific or monospecific (Zapata et al. Protein Eng. 8:1057-62 (1995)).

A “minibody” consisting of scFv fused to CH3 via a peptide linker (hingeless) or via an IgG hinge has been described in Olafsen, et al., Protein Eng Des Sel. 2004 April; 17(4):315-23.

The term “maxibody” refers to bivalent scFvs covalently attached to the Fc region of an immunoglobulin, see, for example, Fredericks et al, Protein Engineering, Design & Selection, 17:95-106 (2004) and Powers et al., Journal of Immunological Methods, 251:123-135 (2001).

Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H2L2 (referred to as “heavy-chain antibodies” or “HCAbs”). Camelized VHH reportedly recombines with IgG2 and IgG3 constant regions that contain hinge, CH2, and CH3 domains and lack a CH1 domain. Classical VH-only fragments are difficult to produce in soluble form, but improvements in solubility and specific binding can be obtained when framework residues are altered to be more VHH-like. (See, e.g., Reichman, et al., J Immunol Methods 1999, 231:25-38.) Camelized VHH domains have been found to bind to antigen with high affinity (Desmyter et al., J. Biol. Chem. 276:26285-90, 2001) and possess high stability in solution (Ewert et al., Biochemistry 41:3628-36, 2002). Methods for generating antibodies having camelized heavy chains are described in, for example, in U.S. Patent Publication Nos. 2005/0136049 and 2005/0037421. Alternative scaffolds can be made from human variable-like domains that more closely match the shark V-NAR scaffold and may provide a framework for a long penetrating loop structure.

Because the variable domain of the heavy-chain antibodies is the smallest fully functional antigen-binding fragment with a molecular mass of only 15 kDa, this entity is referred to as a nanobody (Cortez-Retamozo et al., Cancer Research 64:2853-57, 2004). A nanobody library may be generated from an immunized dromedary as described in Conrath et al., (Antimicrob Agents Chemother 45: 2807-12, 2001).

Further examples of appropriate recombinant methods and exemplary DNA constructs useful for recombinant expression of embodiments of antigen binding proteins by mammalian cells, including dimeric Fc fusion proteins (“peptibodies”) or chimeric immunoglobulin (light chain+heavy chain)-Fc heterotrimers (“hemibodies”), conjugated to pharmacologically active toxin peptide analogs of the invention, are found uin, e.g., Sullivan et al., Toxin Peptide Therapeutic Agents, US2007/0071764 and Sullivan et al., Toxin Peptide Therapeutic Agents, PCT/US2007/022831, published as WO 2008/088422, which are both incorporated herein by reference in their entireties.

Peptides

Peptide or polypeptide compositions for use in the present invention can be made using recombinant DNA technologies, as previously described herein, or by chemical peptide synthesis. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Suitable techniques are well known in the art. (E.g., Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527; “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183). For further examples of synthetic and purification methods known in the art, which are applicable to making the inventive compositions of matter, see, e.g., Sullivan et al., Toxin Peptide Therapeutic Agents, US2007/0071764 and Sullivan et al., Toxin Peptide Therapeutic Agents, PCT/US2007/022831, published as WO 2008/088422 A2, which are both incorporated herein by reference in their entireties.

Linkers.

A “linker” or “linker moiety”, as used interchangeably herein, refers to a biologically acceptable peptidyl or non-peptidyl organic group that is covalently bound to an amino acid residue of a polypeptide chain (e.g., an immunoglobulin HC or immunoglobulin LC or immunoglobulin Fc domain) contained in the inventive composition, which linker moiety covalently joins or conjugates the polypeptide chain to another molecule or chemical moiety. Many useful peptidyl and non-peptidyl linkers are known in the art, and many are described in detail in Sullivan et al., Toxin Peptide Therapeutic Agents, US 2007/0071764 A1. The presence of any linker moiety in the components or reagents employed in the present invention is optional. When present, the linker's chemical structure is not critical, since it serves primarily as a spacer to position, join, connect, or optimize presentation or position of one functional moiety in relation to one or more other functional moieties.

The invention is illustrated by the following examples, which are not intended to be limiting in any way.

### EXAMPLES

### Example 1

### Screening Assay for Neutralizing Antibodies Against KW-0761

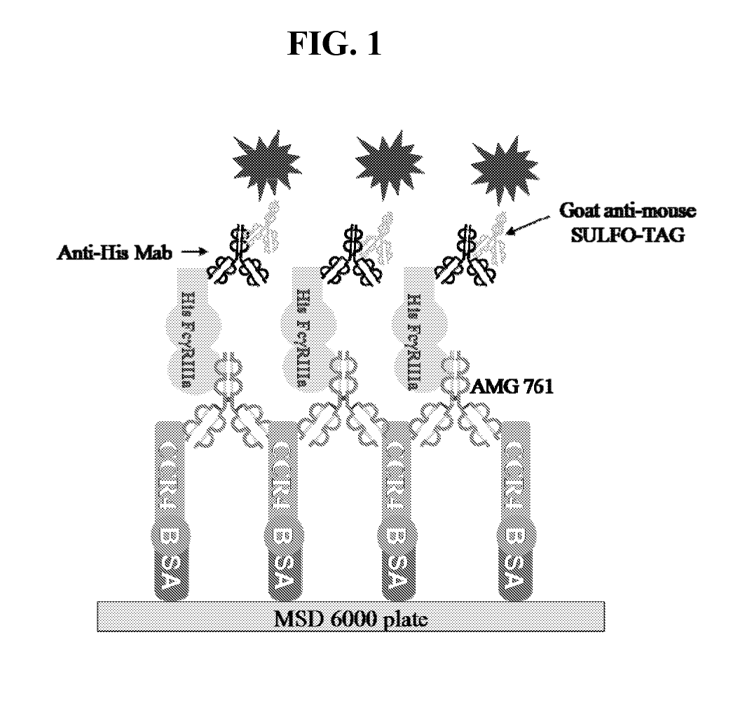

The KW-0761 (also known as “mogamulizumab” or “AMG 761”) therapeutic drug in development is a humanized IgG1 anti-CCR4 antibody which functions through the mechanism of antibody dependent cell-mediated cytotoxicity (ADCC) in vivo. (Ishii et al., Defucosylated Humanized Anti-CCR4Monoclonal Antibody KW-0761 as a Novel Immunotherapeutic Agent for Adult T-cell Leukemia/Lymphoma, Clinical Cancer Research 16: 1520-31 (2010); Shitara et al., Human CDR-grafted antibody and antibody fragment thereof, U.S. Pat. No. 7,504,104). The drug binds to the human CCR4 chemokine receptor on the target cell and human FCγRIIIa (CD16a) on the effector cell. As a result, the effector cell is able to kill the target cell. We developed an embodiment of the dual function target binding assay for the detection of neutralizing antibodies (NAb) against KW-0761 by utilizing the MSD electrochemiluminescence (ECL) detection method. FIG. 1 shows a schematic representation of an embodiment of the inventive assay, as configured for detecting an IgG1 target antibody (e.g., KW-0761) that specifically binds a biotinylated target protein of interest (e.g., CCR4), and for detecting neutralizing antibodies in a serum sample.

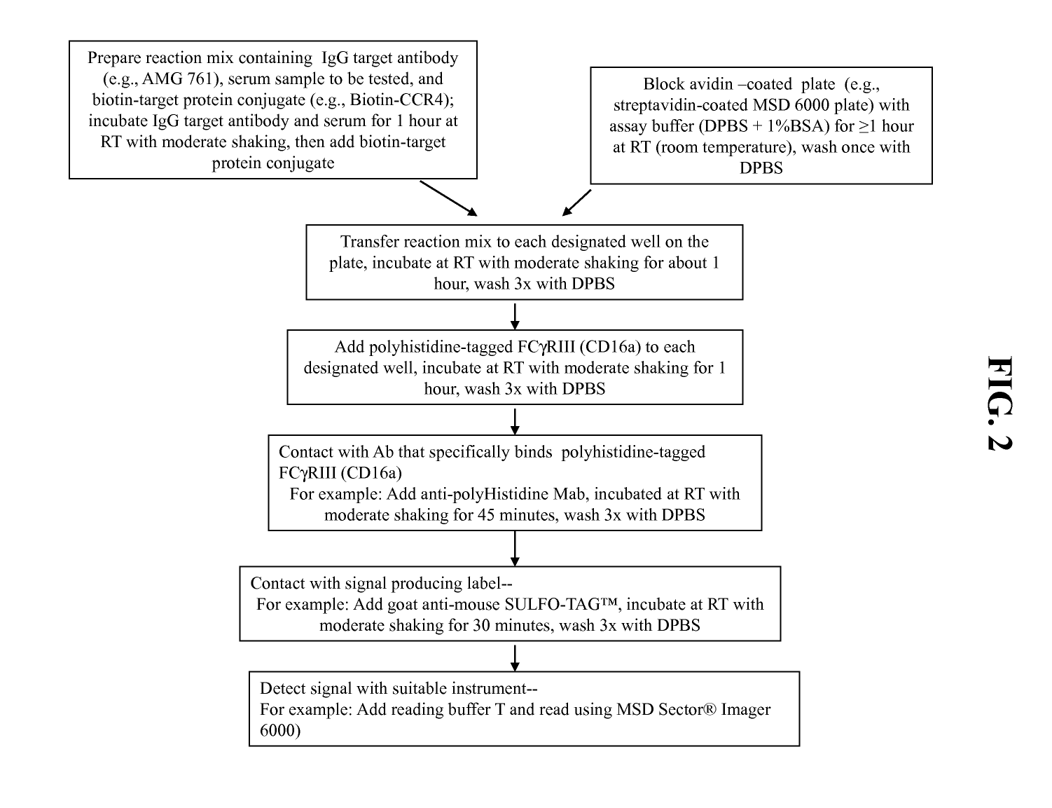

Briefly, in this embodiment of the present invention, a reaction mixture containing the IgG target antibody (the therapeutic drug, e.g., KW-0761; Ishii et al., Defucosylated Humanized Anti-CCR4 Monoclonal Antibody KW-0761 as a Novel Immunotherapeutic Agent for Adult T-cell Leukemia/Lymphoma, Clinical Cancer Research 16: 1520-31 (2010); Shitara et al., Human CDR-grafted antibody and antibody fragment thereof, U.S. Pat. No. 7,504,104), a serum sample to be tested, and a polypeptide portion of a target protein (e.g., biotin-CCR4 peptide conjugate) is added to streptavidin-coated wells in a plate (e.g., a 96-well streptavidin-coated plate, MSD catalog #L11SA-1, Meso Scale Discovery [MSD], Gaithersburg, Md.). After the reaction mixture is incubated on the plate, the plate is washed before the addition of polyhistidine-tagged recombinant human FCγRIIIa (CD16a), followed by incubation with an anti-polyhistidine mouse monoclonal antibody; anti-polyhistidine monoclonal antibody from a different animal species can also be used, if convenient. Finally, electrochemiluminescence (ECL) signal is generated with the addition of Sulfo-TAG™-labeled goat anti-mouse antibody (MSD catalog #R32AC-1; labeled antibody from a different animal species can also be used, if convenient, as long as the antibody specifically reacts with the anti-polyhistidine antibody that is used) and Read Buffer T obtained from MSD (MSD catalog #R92TC-1). FIG. 2 shows a flowchart of assay method steps in this embodiment of the invention. If the serum sample contains neutralizing antibodies against the IgG target antibody, the IgG target antibody will not be able to bind to the biotin-CCR4 peptide that is captured on the streptavidin-coated well surfaces. Consequently, a low ECL signal is generated in the presence of NAb. In the absence of NAb, a high ECL signal is generated because the drug is able to build a bridge between the biotin-CCR4 peptide and the histidine tagged recombinant human FCγRIIIa (CD16a). If the screening assay indicates the presence of NAbs, then a confirmatory assay, such as the one described in Example 2 herein is recommended.

Screening Assay Protocol.

The following detailed assay protocol was carried out:

1. Streptavidin-coated wells in a 96-well plate (MSD catalog #L11SA-1, Meso Scale Discovery, Gaithersburg, Md.) were each blocked with 150 μL of assay buffer (Dulbecco's Phosphate Buffered Saline [pH 7.1±0.1; “DPBS”; obtained from Invitrogen]+1% (v/v) bovine serum albumin [“BSA”]) for each well for at least 1 hour at ambient temperature to ensure adequate blocking of the wells. Blocking may be done for longer than an hour, if convenient, as long as the blocked wells are used in the inventive assay on the same day. The wells were washed once with 200 μL/well DPBS after blocking. The assay buffer chosen was minus calcium or magnesium dications, however these dications are not believed to interfere with the inventive assay.
2. Reaction mixture (50 μL/well) was prepared containing IgG target antibody KW-0761 (20 ng/mL final concn.), serum sample (10% (v/v) final concn), and biotin-CCR4 peptide conjugate (80 ng/mL final concn.) in assay buffer, with volumes adjusted to suffice for the desired number of wells to which the reaction mixture needs to be transferred in step 3 below.
(a) The serum sample: 10 μL of 50% (v/v) serum sample +30 μL of 34 ng/mL-KW-0761 diluted in assay buffer.
(b) The following control samples were also made:
“N” control: 10 μL of 50% (v/v) pooled human serum [from normal donors] (“PHS”; obtained from Bioreclamation, Inc., Long Island, N.Y.)+30 μL assay buffer; or
“D” control: 10 μL of 50% (v/v) PHS+30 μL of 34 ng/mL KW-0761 diluted in assay buffer; or
“P” control: 10 μL of 50% (v/v) PHS spiked with anti-KW-0761 antibody (rabbit anti-KW-0761 polyclonal stock at concn of 1.02 mg/mL stored at −70° C.)+30 μL of 34 ng/mL KW-0761 diluted in assay buffer.
Dilution to 50% (v/v) of PHS in these controls or the serum sample was accomplished by combining with an equal volume of assay buffer. The samples in #2(a) or #2(b) above were incubated in a U- or V-bottom polypropylene plate with moderate shaking for 1 hour (±15 minutes) at ambient temperature, after which:
(c) The biotin-CCR4 peptide conjugate (10 μL of 400 ng/mL biotin-CCR4 peptide diluted in assay buffer prepared from a 1 mg/mL-stock solution stored at −70° C.; once thawed, stock was kept at 4° C. for no more than 1 month) was aliquoted into the samples in #2(a) or #2(b) above to form the reaction mixture. The biotinylated polypeptide portion of CCR4 had the amino acid sequence: MNPTDIADTTLDESIYSNYYLYESIPKPK(BIOTIN)-OH//SEQ ID NO:3 (purchased from Midwest Bio-Tech Inc., catalog #MBT3898); or alternatively, MNPTDIADTTLDESIYSNYYLYESIPKPCTK(Biotin)-OH//SEQ ID NO:4, which was synthesized and biotinylated by conventional chemical techniques.
3. The reaction mixture (including serum sample or control sample(s)) from step #2 above was transferred to each designated well on the streptavidin-coated plate from step #1 above (MSD catalog #L11SA-1, Meso Scale Discovery, Gaithersburg, Md.). Each well received 50 μL of the reaction mixture. The streptavidin-coated plate was then incubated with moderate shaking at ambient temperature for 1 hour (±15 minutes), after which the wells were washed three times with DPBS, 200 μL per well per wash.
4. 50 μL of 80 ng/mL polyhistidine tagged recombinant FCγRIII (obtained from R&D Systems, catalog #4325-FC) diluted in assay buffer were added to each designated well on the streptavidin-coated plate, incubate at ambient temperature with moderate shaking for 1 hour (±15 minutes), after which the wells were washed three times with DPBS, 200 μL per well per wash.
5. 100 μL of 1 μg/mL of mouse anti-polyhistidine Mab (obtained from R&D Systems, catalog #MAB050) diluted in assay buffer were added to each designated well on the streptavidin-coated plate, incubate at ambient temperature with moderate shaking for 45-60 minutes), after which the wells were washed three times with DPBS, 200 μL per well per wash.
6. 100 μL of 1 μg/mL of goat anti-mouse Sulfo-TAG™ (MSD catalog #R32AC-1) diluted in assay buffer were added to each designated well on the MSD plate, incubate at ambient temperature with moderate shaking for 30-45 minutes (the plate was covered with foil during incubation), after which the wells were washed three times with DPBS, 200 μL per well per wash.
7. 150 μL of 1× Read Buffer T (MSD catalog #R92TC-1; diluted to 1× from 4× stock using water) were added to each well and read with a SECTOR® Imager 6000 reader (“MSD 6000”; Meso Scale Discovery, Gaithersburg, Md.).

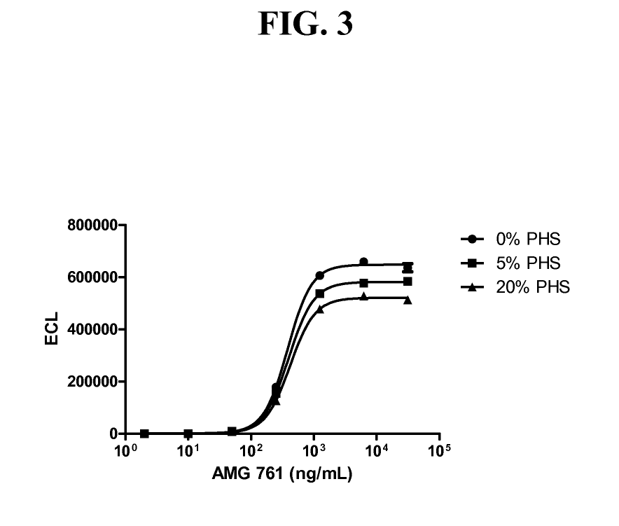

A representative dose response for KW-0761 in the inventive assay in the presence of assay buffer (0% PHS) or pooled human serum (PHS: 5% PHS or 20% PHS; (v/v)) is shown in FIG. 3.

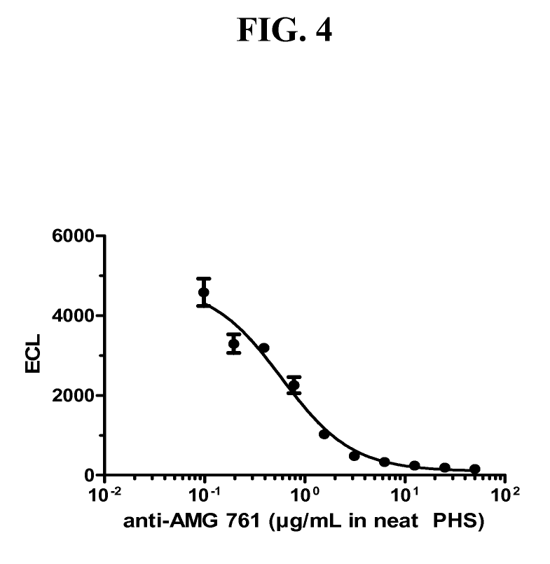

FIG. 4 demonstrates that binding of KW-0761 to the biotinylated CCR4 peptide is inhibited by the presence of polyclonal anti-KW-0761 neutralizing antibodies in a dose dependent manner.

### Example 2

### General Protein G/L Depletion Protocol for Confirmatory Assay

If the inventive screening assay, e.g., as described in Example 1 herein, indicates the presence of neutralizing antibodies in a serum sample, then a sensitive confirmatory assay is recommended. The following confirmatory assay protocol employs serum sample filtrate from a Protein G/L incubation in the screening assay protocol described in Example 1 herein:

1. Protein G agarose resin (Pierce catalog #20520) and Protein L agarose resin (Pierce catalog #22851) were mixed in equal portions then the resin mixture was diluted with Dulbecco's Phosphate Buffered Saline (pH 7.1±0.1; “DPBS”; obtained from Invitrogen) to create a 50% bead slurry (“Protein G/L”). (A large volume of the 50% bead slurry can be prepared beforehand as a reagent, given a 3 month expiry date and stored at 2° to 8° C.)
2. Sepharose 6B resin (Sigma catalog #6B100) was diluted with DPBS to create 50% bead slurry (“Sepharose 6B”). (A large volume of the 50% bead slurry can be prepared beforehand as a reagent, given a 3 month expiry date and stored at 2° to 8° C.) Sepharose 6B treatment is performed as a control for each sample that is treated with the Protein G/L resin. NAbs present in the serum samples can be removed by Protein G/L resin treatment, but not Sepharose 6B resin treatment.
3. Maintaining the resin beads continuously in suspension, 120 μL of either Protein G/L (prepared in #1 above) or Sepharose 6B (prepared in #2 above) were loaded into appropriate wells of a multiscreen filter plate (e.g., Millipore catalog #MSHVS4510).
4. A recipient 96-well plate was placed beneath the filter plate (prepared in #3 above), and the filter plate was washed twice (200 μL/well/wash) with DPBS using centrifugation (1000-2000 RPM). All flow-through from the filter plate was collected and discarded. The final centrifugation should leave relatively dry resin pellets in the wells.
5. Serum samples to be tested were diluted to 50% (v/v) by combining a volume of each serum sample with an equal volume of assay buffer (Dulbecco's Phosphate Buffered Saline [pH 7.1±0.1; “DPBS”; obtained from Invitrogen]+1% (v/v) bovine serum albumin [“BSA”]).
6. Each diluted serum sample was added to a corresponding Protein G/L pellet and a Sepharose 6B pellet. The filter plate wells were covered with a lid or top seal.
7. A fresh recipient 96-well plate was placed beneath the filter plate as a recipient plate, and the recipient plate/filter plate combination was vigorously mixed using a plate shaker or similar device for at least 30 minutes. After 30 minutes of mixing, the recipient plate/filter plate combination was centrifuged at 1000-2000 RPM to collect the filtrate in the recipient plate.
8. The filtrate collected in #7 above was used directly in the confirmatory assay, the steps of which were the same as those in the screening assay procedures described in Example 1 herein. For each reaction mixture, 10 μL of the filtrate from the Protein G/L treatment (or Sepharose 6B filtrate control) should be added to 30 μL of 34 ng/mL of KW-0761 (also known as “mogamulizumab” or “AMG 761”) diluted in assay buffer. After the filtered serum sample and drug mix is incubated (as in Example 1, step #2(a) hereinabove), 10 μL of 400 ng/mL of biotin-CCR4 is added to make the complete reaction mixture (as in Example 1, step #2(c) hereinabove). The rest of steps #3-7 in Example 1 were carried out.

### Example 3

### Rituximab Target Binding Assay

Rituximab (available from Genentech, a member of the Roche Group, as Rituxan®) is a monoclonal IgG1 therapeutic antibody which selectively targets CD20⁺ B cells. (Hauser et al., B-cell depletion with rituximab in relapsing-remitting multiple sclerosis, NEJM 358:676-88 (2008)). One of the proposed mechanisms of action of rituximab is ADCC. The target binding assay format developed for KW-0761, as described in Example 1 herein, can be adapted for rituximab, which has a CD16a binding site in its Fc domain, by: (i) replacing KW-0761 in Example 1, step #2(a)-(b) hereinabove with similar quantities of rituximab; (ii) replacing rabbit anti-KW-0761 antibody in Example 1, step #2(b) with similar quantities of rabbit anti-rituximab antibody; and (iii) replacing biotinylated-CCR4 peptide conjugate in Example 1, step #2(c) hereinabove with similar quantities of a biotin-CD20 peptide conjugate (e.g., MBT4736: (Biotin)-[Ahx]KGGYNCEPA NPSEKNSPST QYCYS IQSL//SEQ ID NO:7; or MBT4737: YNCEPANPSEKNSPSTQYCYSIQSLK[Ahx] K(Biotin) NH₂//SEQ ID NO:8; both purchased from Midwest Bio-Tech Inc.; in other embodiments, (Biotin)KGGYNCEPANPSEKNSPSTQYCYSIQSL//SEQ ID NO:5 or YNCEPANPSEKNSPSTQYCYSIQSLKK(Biotin)//SEQ ID NO:6 were used instead, which were synthesized and biotinylated by conventional chemical techniques). All other steps in Example 1 (#3-7) are directly adaptable without substantial modification.

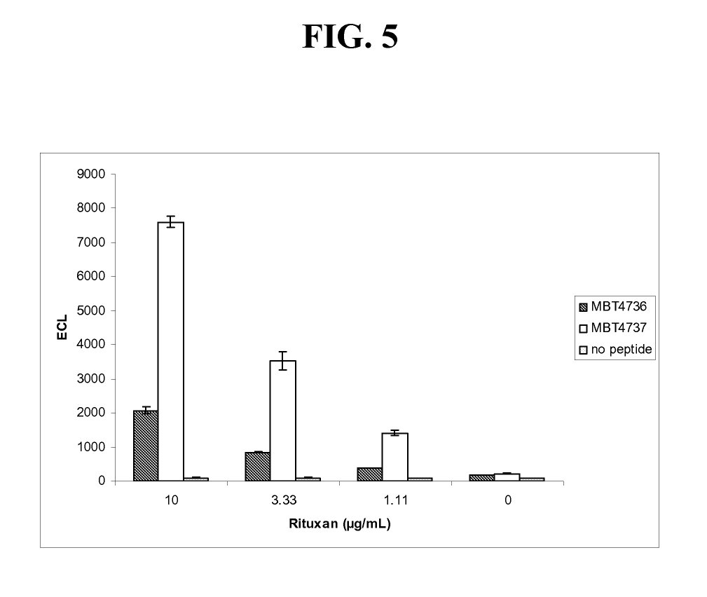

An experiment was run in the same assay format described in Example 1, but modified in that a human serum sample was absent and any PHS called for was replaced with assay buffer (DPBS [pH 7.1±0.1]+1% [v/v] bovine serum albumin), the “N” control containing PHS diluted in assay buffer was absent, the “P” control with anti-rituximab antibody was absent, and the “D” control was run at multiple concentrations of rituximab so as to allow titration of the rituximab in the assay method (see, FIG. 5). Polyhistidine tagged recombinant human FCγRIIIa/CD16a was added to bind to the biotin-CD20 peptide (SEQ ID NO:7 or SEQ ID NO:8)/rituximab complex that was captured on the streptavidin-coated plate (see, Example 1, step #1). The inventive screening assay was further conducted in all other respects as in Example 1 herein. Briefly, ECL signal was generated with the addition of an anti-polyhistidine monoclonal antibody, followed by the Sulfo-TAG™-labeled goat anti-mouse antibody and Read Buffer T and signal was detected with a SECTOR® Imager 6000 reader (“MSD 6000”; Meso Scale Discovery, Gaithersburg, Md.), as described in Example 1. FIG. 5 shows representative data from the experiment, demonstrating that rituximab bound to the biotinylated CD20 peptide conjugate and was detectable in a dose-dependent manner in the same assay format described in Example 1, but modified as described in this Example 3.

What is claimed:

1. An in vitro assay method, comprising:
detecting in an avidin-coated well by measuring a signal, in the presence of a fresh volume of a buffer permitting detection under physiological conditions, any of an antibody that specifically binds polyhistidine, that has bound a polyhistidine-tagged recombinant human CD16a polypeptide, wherein the avidin-coated well was previously blocked and subsequently a pre-incubated reaction mixture has been incubated in the blocked avidin-coated well, under physiological conditions, wherein the pre-incubated reaction mixture was suspended during its pre-incubation in an aqueous serum-containing assay buffer, and the pre-incubated reaction mixture comprised:
(i) a target antigen binding protein comprising an Fc domain with a CD16a binding site; and
(ii) a biotinylated polypeptide portion of a target protein of interest, to which polypeptide portion the target antigen binding protein specifically binds; and
wherein, subsequent to the incubation of the pre-incubated reaction mixture in the avidin-coated well, a polyhistidine-tagged recombinant human CD16a polypeptide suspended in a fresh volume of the assay buffer was incubated under physiological conditions, together with any target antigen binding protein that was bound to the biotinylated polypeptide portion that was bound to the avidin of the avidin-coated well; and wherein, prior to detecting by measuring the signal, the antibody that specifically binds polyhistidine, suspended in a fresh volume of the assay buffer, was incubated in the well under physiological conditions, together with any polyhistidine-tagged recombinant human CD16a polypeptide that was bound to the target antigen binding protein.

2. The method of claim 1, further comprising before detecting the antibody that specifically binds polyhistidine, the step of incubating in the well, under physiological conditions, an antibody that comprises a conjugated signal-producing label, suspended in a fresh volume of the assay buffer, wherein said antibody specifically binds the antibody that specifically binds polyhistidine.

3. The method of claim 1, wherein the pre-incubated reaction mixture further comprised a serum sample to be tested for the presence of neutralizing antibodies.

4. The method of claim 1, wherein the target antigen binding protein is an IgG1 or IgG3 antibody.

5. The method of claim 1, wherein the target protein of interest is CCR4.

6. The method of claim 1, wherein the target protein of interest is CD20.

7. The method of claim 1, wherein the target protein of interest is HER2.

8. The method of claim 1, wherein the avidin is streptavidin.

9. The method of claim 1, wherein the antibody that specifically binds polyhistidine further comprises a conjugated signal-producing label.

10. The method of claim 8, wherein the signal-producing label comprises a fluorescent label, an isotopic label, an electrochemiluminescent label, or an enzyme.

11. An in vitro assay method, comprising:
(a) incubating in a blocked avidin-coated well, under physiological conditions, a pre-incubated reaction mixture suspended in an aqueous serum-containing assay buffer, the pre-incubated reaction mixture comprising:
(iii) an IgG target antibody comprising an Fc domain with a CD16a binding site; and
(iv) a biotinylated polypeptide portion of a target protein of interest, to which polypeptide portion the IgG target antibody specifically binds;
(b) incubating in the well, under physiological conditions, a polyhistidine-tagged recombinant human CD16a polypeptide suspended in a fresh volume of the assay buffer, together with any IgG target antibody that was bound to the biotinylated polypeptide portion that was bound to the avidin in (a);
(c) incubating in the well, under physiological conditions, an antibody that specifically binds polyhistidine, said antibody suspended in a fresh volume of the assay buffer, together with any polyhistidine-tagged recombinant human CD16a polypeptide that was bound to the IgG target antibody in (b); and
(d) detecting in the well, in the presence of a fresh volume of a buffer permitting detection under physiological conditions, any of the antibody that specifically binds polyhistidine that was bound to the polyhistidine-tagged recombinant human CD16a polypeptide in (c) by measuring a signal.

12. The method of claim 11, wherein the pre-incubated reaction mixture further contains a serum sample to be tested for the presence of neutralizing antibodies.

13. The method of claim 11, wherein the IgG target antibody is an IgG1 or IgG3 antibody.

14. The method of claim 11, wherein the target protein of interest is CCR4.

15. The method of claim 11, wherein the target protein of interest is CD20.

16. The method of claim 11, wherein the target protein of interest is HER2.

17. The method of claim 11, wherein the avidin is streptavidin.

18. The method of claim 11, wherein the antibody that specifically binds polyhistidine further comprises a conjugated signal-producing label.

19. The method of claim 18, wherein the signal-producing label comprises a fluorescent label, an isotopic label, an electrochemiluminescent label, or an enzyme.

20. The method of claim 11, further comprising before step (d) the step of incubating in the well, under physiological conditions, an antibody that comprises a conjugated signal-producing label, suspended in a fresh volume of the assay buffer, wherein said antibody specifically binds the antibody that specifically binds polyhistidine in (c).

21. The method of claim 20, wherein the signal-producing label comprises an electrochemiluminescent label.

22. The method of claim 21, wherein the electrochemiluminescent label comprises a ruthenium complex.

23. The method of claim 22, wherein the electrochemiluminescent label is formed from an N-hydroxysucccinimide ester.

24. The method of claim 23, wherein the N-hydroxysuccinimide ester has the chemical structure:

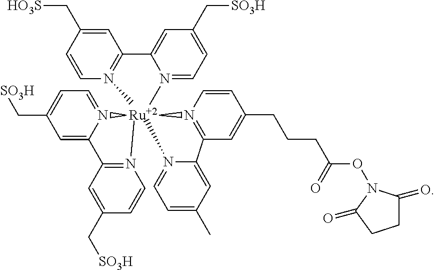

25. The method of claim 22, wherein the electrochemiluminescent label comprises a ruthenium complex having the following formula, wherein the line drawn from the carbonyl group shows the attachment to the rest of the molecule:

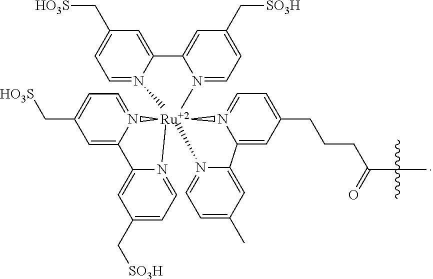

26. The method of claim 20, wherein the signal-producing label comprises a fluorescent label, an isotopic label, or an enzyme.

27. The method of claim 14, wherein the IgG target antibody is mogamulizumab.

28. The method of claim 15, wherein the IgG target antibody is rituximab.

29. The method of claim 16, wherein the IgG target antibody is trastuzumab.

30. An in vitro assay method, comprising:
(a) incubating in a blocked avidin-coated well, under physiological conditions, a pre-incubated reaction mixture suspended in an aqueous serum-containing assay buffer, the pre-incubated reaction mixture comprising:
(i) an IgG target antibody against human CCR4 comprising an Fc domain with a CD16a binding site;
(ii) serum sample to be tested for the presence of neutralizing antibodies; and
(iii) a biotinylated polypeptide portion of human CCR4, to which polypeptide portion the IgG target antibody specifically binds;
(b) incubating in the well, under physiological conditions, a polyhistidine-tagged recombinant human CD16a polypeptide suspended in a fresh volume of the assay buffer, together with any IgG target antibody that was bound to the biotinylated polypeptide portion of human CCR4 that was bound to the avidin in (a);
(c) incubating in the well, under physiological conditions, an antibody that specifically binds polyhistidine, said antibody suspended in a fresh volume of the assay buffer, together with any polyhistidine-tagged recombinant human CD16a polypeptide that was bound to the IgG target antibody in (b);
(d) incubating in the well, under physiological conditions, an antibody that comprises a conjugated signal-producing label, suspended in a fresh volume of the assay buffer, wherein said antibody specifically binds the antibody that specifically binds polyhistidine in (c); and
(e) detecting in the well, in the presence of a fresh volume of a buffer permitting detection under physiological conditions, any signal produced.

31. The method of claim 30, wherein the signal-producing label comprises an electrochemiluminescent label.

32. The method of claim 31, wherein the electrochemiluminescent label comprises a ruthenium complex.

33. The method of claim 32, wherein the electrochemiluminescent label is formed from an N-hydroxysucccinimide ester.

34. The method of claim 33, wherein the N-hydroxysuccinimide ester has the chemical structure:

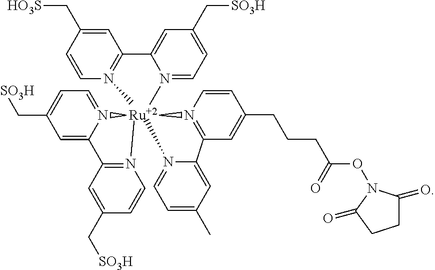

35. The method of claim 32, wherein the electrochemiluminescent label comprises a ruthenium complex having the following formula, wherein the line drawn from the carbonyl group shows the attachment to the rest of the molecule:

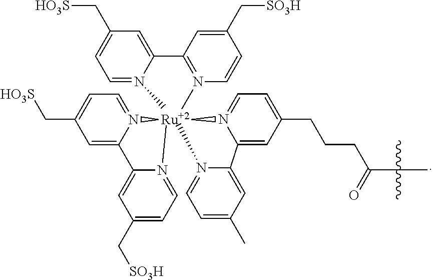

36. The method of claim 30, wherein the signal-producing label comprises a fluorescent label, an isotopic label, or an enzyme.

37. The method of claim 30, wherein the avidin is streptavidin.

38. The method of claim 30, wherein the IgG target antibody is mogamulizumab.
